(19) United States
(12) Patent Application Publication
(10) Pub. No.: US 2025/0107923 A1
(43) Pub. Date: Apr. 3, 2025

(54) DEVICE FOR PROVIDING HEATING AND COOLING THERAPY TO A SHOULDER

(71) Applicant: HYPERICE IP SUBCO, LLC, Irvine, CA (US)

(72) Inventors: Alexander Joseph Aguiar, San Diego, CA (US); Trevor Austin Kerth, San Diego, CA (US); Robert Glen Edwards, San Diego, CA (US)

(21) Appl. No.: 18/896,087

(22) Filed: Sep. 25, 2024

Related U.S. Application Data
(60) Provisional application No. 63/586,945, filed on Sep. 29, 2023.

(51) Int. Cl. A61F 7/00; A61H 23/02

(52) U.S. Cl. CPC A61F 7/007; A61F 2007/003; A61F 2007/0075; A61F 2007/0078; A61F 2007/0093; A61H 23/02

(57) ABSTRACT

A wearable system or apparatus for applying thermal therapy to a shoulder includes a number of temperature modulation devices arranged in a selected pattern. The pattern can be selected such that when the wearable system or apparatus is disposed over a shoulder, the temperature modulation devices can be symmetrically disposed over the front and the back of the shoulder. In some examples, the temperature modulation devices can provide heat and/or a cooling effect to the shoulder region.

### CROSS-REFERENCE TO RELATED APPLICATIONS

This application claims priority and benefit from U.S. Provisional Application No. 63/586,945, titled “Device for Providing Heating and Cooling Therapy to a Shoulder” and filed on Sep. 29, 2023, which are hereby incorporated by reference herein in its entirety.

### TECHNICAL FIELD

The present disclosure relates generally to the therapy field, and more specifically to devices and methods for delivering heating and/or cooling therapy.

### BACKGROUND

Conventional temperature therapy devices often provide only one kind of therapy. For example, an electric heating pad or blanket can only provide heat therapy. On the other hand, some cooling pads or blankets use ice, cold water, or thermoelectric coolers (TECs), and provide only the cooling therapy. Among these devices, those that have a generic shape, such as a pad or blanket, can be used on different parts of the body, such as lower legs, knees, arms, chest, back, shoulders, and/or neck. But, the heating and/or cooling elements in such pads and blankets are not specifically disposed according the shape of any particular body part to which therapy is to be provided. As such, these devices may not be effective in providing heating and/or cooling therapy to a particular body part, e.g., the leg or the shoulder. While some devices are designed for specific body parts, such as the neck or the shoulders, the use of ice and/or cold water in such devices makes the device bulky, cumbersome to use, and may also limit the duration of the use.

Some devices, typically blankets, can provide both heating and cooling effects, e.g., by circulating cool air for cooling, and by using one or more heating elements for heating. In some devices, the blanket itself may be frozen, in order to provide the cooling therapy. Generating and circulating cool air can make such devices bulky, and having to freeze the blanket makes its use cumbersome. Moreover, like the heating only or cooling only devices, many of these devices can also be ineffective, because the heating and/or cooling elements in such pads and blankets are not specifically disposed according the shape of any particular body part to which therapy is to be provided. Additionally, due to their bulkiness, many of the conventional devices are not well suited for travel.

### SUMMARY

Temperature therapy (e.g., hot and cold therapy) (also called thermal therapy) has been shown to be effective in injury recovery, helping to expedite the healing process while reducing pain, inflammation, and joint stiffness. Localized cooling can induce vasoconstriction with reflexive vasodilation and/or reduce bleeding, inflammation, metabolism, muscle spasm, pain, enzymatic activity, oxygen demand, and/or swelling in areas of the body affected by soft tissue trauma or injury. Localized heating can increase blood flow, decrease sensation of pain, increase local tissue metabolic rate, increase the rate of healing, and/or facilitate the stretching of tissue. In some cases, a combination of heat and cold therapies (also called treatments), where heating and cooling may be applied to a part of the body intermittently, can be more effective than applying only the heat treatment or only the cold treatment. Furthermore, due to its peculiar shape, applying heat and/or cold treatment to the shoulder can be more challenging using a pad or a blanket.

Therefore, described herein is a head and cold treatment device that is specifically designed according to shape of a shoulder. The device can selectively provide both heat and cooing treatment to the shoulder. In some embodiments, the device can also provide vibration forces to the shoulder in the alternative, or in combination with heat or cold treatment.

Accordingly, in one aspect, an apparatus is disclosed for providing thermal therapy to a shoulder. The apparatus includes a support layer that has an axis that can be aligned with a collarbone of a shoulder. The apparatus also includes a number of temperature modulation devices mounted to the support layer, where the devices are mounted in a symmetrical pattern about the axis. Additionally, the apparatus includes a controller for controlling a temperature of at least one of the temperature modulation devices.

The temperature modulation devices may include a set of cooling elements. Alternatively, the temperature modulation devices may include: (i) a first set of heating elements, and (ii) a second, different set of cooling elements. One or more of the set of cooling elements may include a thermoelectric cooling (TEC) device. In some embodiments, the temperature modulation devices include one or more heating-cooling device that can selectively provide heat or a cooling effect.

In some embodiments, the apparatus includes a base having several pleats, wherein at least a subset of the number of temperature modulation devices are disposed in respective pleats of the several pleats. In other words, the pleats may form valleys in the base, where a respective modulation device may be disposed in a respective valley. The apparatus may also include a battery for supplying electrical power to the controller and to one or more of the number of temperature modulation devices. The apparatus may include a first set of loops and an associated first strap that can be passed through the first set of loops and wrapped around a torso. The apparatus may also include a second set of loops and an associated second strap. The second strap may be passed through the second set of loops and may be wrapped around an upper arm. When the straps are wrapped in this manner, the axis of the support layer may align substantially with the collarbone of a shoulder over which the apparatus is placed. In some embodiments, the apparatus may include a number of vibration generation devices mounted to the support layer, where the vibration generation devices are mounted in a symmetrical pattern about the axis.

The above and other preferred features, including various novel details of implementation and combination of events, will now be more particularly described with reference to the accompanying figures and pointed out in the claims. It will be understood that the particular systems and methods described herein are shown by way of illustration only and not as limitations. As will be understood by those skilled in the art, the principles and features described herein may be employed in various and numerous embodiments without departing from the scope of any of the present inventions. As can be appreciated from foregoing and following description, each and every feature described herein, and each and every combination of two or more such features, is included within the scope of the present disclosure provided that the features included in such a combination are not mutually inconsistent. In addition, any feature or combination of features may be specifically excluded from any embodiment of any of the present inventions.

### BRIEF DESCRIPTION OF THE DRAWINGS

The accompanying figures illustrate various embodiments. Together with the general description given above and the detailed description given below, these figures explain the principles described herein. In general, the same reference numeral across different figures refers to or identifies the same component.

FIGS. 1A and 1B schematically depict an apparatus for providing cooling therapy to a shoulder, according to one embodiment.

FIGS. 2A and 2B schematically depict an apparatus for providing heating and/or cooling therapy to a shoulder, according to another embodiment.

FIG. 3 depicts a temperature modulation device that may be included in an apparatus for providing heating and/or cooling therapy to a shoulder, according to one embodiment.

FIG. 4 schematically depicts a thermoelectric cooling (TEC) device, according to one embodiment;

FIGS. 5A and 5B depict another temperature modulation device that may be included in an apparatus for providing heating and/or cooling therapy to a shoulder, according to one embodiment.

FIGS. 6A and 6B depict a vibration generation device that may be included in an apparatus for providing heating and/or cooling therapy to a shoulder, according to one embodiment.

While the present disclosure is subject to various modifications and alternative forms, specific embodiments thereof have been shown by way of example in the drawings and will herein be described in detail. The present disclosure should be understood to not be limited to the particular forms disclosed, but on the contrary, the intention is to cover all modifications, equivalents, and alternatives falling within the spirit and scope of the present disclosure.

### DETAILED DESCRIPTION

Generally, the invention relates to devices, systems, and methods that include components or modules that can deliver temperature (e.g., hot, cold, and/or a desired temperature) therapy to the shoulders of a user. The devices, systems, and methods may also deliver vibration therapy. In some embodiments, temperature therapy as described herein is applied using a thermoelectric cooler (TEC) as described in: (i) U.S. Patent Publication No. 2023/0329902, titled “Wearable Therapy Device for Providing Temperature and Compression Therapy”; (ii) U.S. Patent Application Publication No. 2019/0350752, titled “Wearable Temperature Therapy System and Method”; (iii) U.S. Pat. No. 11,602,454, titled “Temperature Modulation Assembly and a Multi-Layer Retention Mechanism for a Temperature Therapy Device”; and (iv) U.S. Pat. No. 11,564,831, titled “System and Methods for Monitoring and/or Controlling Temperature in a Therapy Device,” which are incorporated herein by reference in their entirety.

In some embodiments, temperature therapy as described herein is applied using a polyimide or other type of heater as described, for example, in: (v) U.S. Patent Application Publication No. 2020/0170880, titled “Vibration and Heat Generation Apparatus for Use with Compression Wraps”; (vi) U.S. Patent Application Publication No. 2022/0347010, titled “Wearable Therapeutic Device with Replaceable Pads, and Related Systems and Methods”; and (vii) U.S. Patent Application Publication No. 2023/0084903, titled “System and Apparatus to Apply Vibration, Thermal and Compressive Therapy,” which are incorporated herein by reference in their entirety.

In various embodiments, the system generates a high temperature and/or a low temperature at a contact surface that is in thermal contact with a region of the shoulder of a user. A high temperature, as described herein, is a temperature that is higher than a temperature of the user's body in general, and in particular, higher than the skin temperature of the user's shoulder to which the treatment is to be provided. The high temperature may be set automatically by a control module, or by the user or an entity assisting the user. Heat is generally applied according to the set high temperature to provide the therapeutic benefits of such a temperature (e.g., circulation promotion, muscle relaxation, etc.). As examples, the high temperature can be a temperature between 100.0-120.0° F., 102.0-115.0° F., 108.0-109.0° F., etc., or any other suitable temperature range (e.g., above body temperature).

The low temperature, as described herein, is a temperature that is a temperature that is lower than a temperature of the user's body in general, and in particular, lower than the skin temperature of the user's shoulder to which the treatment is to be provided. The low temperature may also be set automatically by a control module, or by the user or an entity assisting the user. Cold therapy is provided according to the set low temperature to provide the therapeutic benefits of such a temperature (e.g., pain relief, inflammation reduction, circulation modulation, etc.). As examples, the low temperature can be a temperature between 45.0-65.0° F., 49.0-61.0° F., 50.0-51.0° F., etc., or any other suitable temperature range (e.g., at or below body temperature).

Both the high and low temperature can correspond to any suitable actual or perceived (e.g., wet bulb) temperature within or outside the aforementioned ranges. In various embodiments, the system can control the high and/or low temperature to within one tenth of one degree Fahrenheit. Additionally or in the alternative, the system may control the high and/or low temperatures at any other suitable precision. The temperature ranges above are typical, but do not necessarily specify the maximum and/or minimum temperature values that may be set and attained. Every value within the respective range provided for is contemplated and expressly supported herein, subject to the number of significant digits expressed in each particular range.

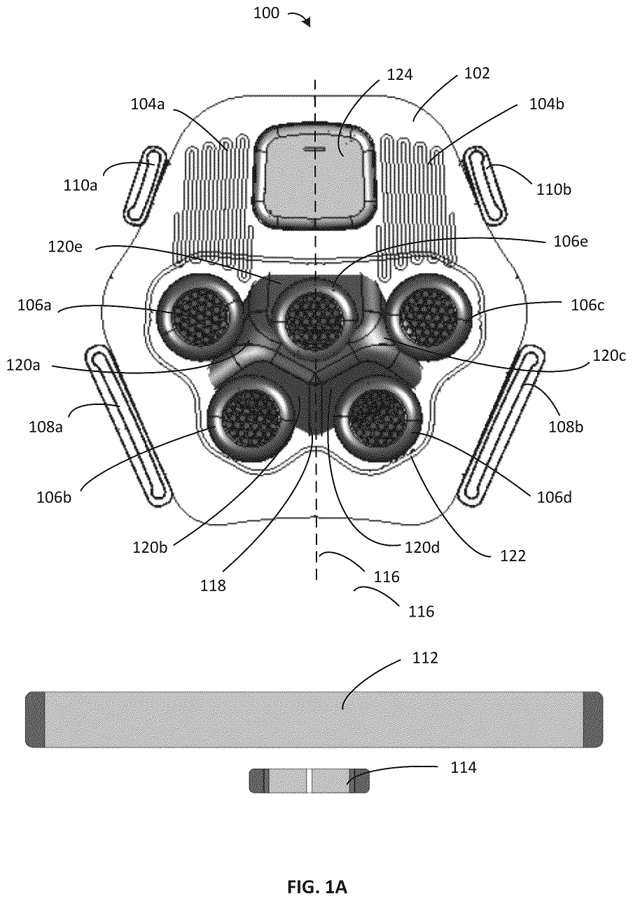

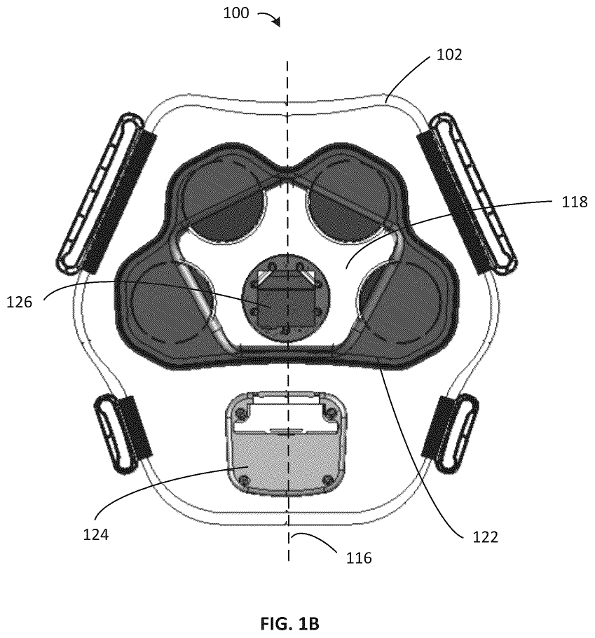

FIG. 1A schematically depicts a shoulder treatment apparatus 100. FIG. 1B depicts the apparatus 100 with certain components eliminated, to expose certain other components. The apparatus 100 includes a support layer 102 that includes patterns 104a, 104b (collectively, patterns 104), and cooling elements 106a-106e (collectively, cooling elements 106). Although the apparatus 100 includes two patterns, other embodiments may include only one, or more (e.g., 3, 4, 6, etc.) patterns. Likewise, although the apparatus 100 includes five cooling elements, other embodiments may include fewer (e.g., only one, two, three, or four), or more (e.g., 6, 9, 10, etc.) cooling elements.

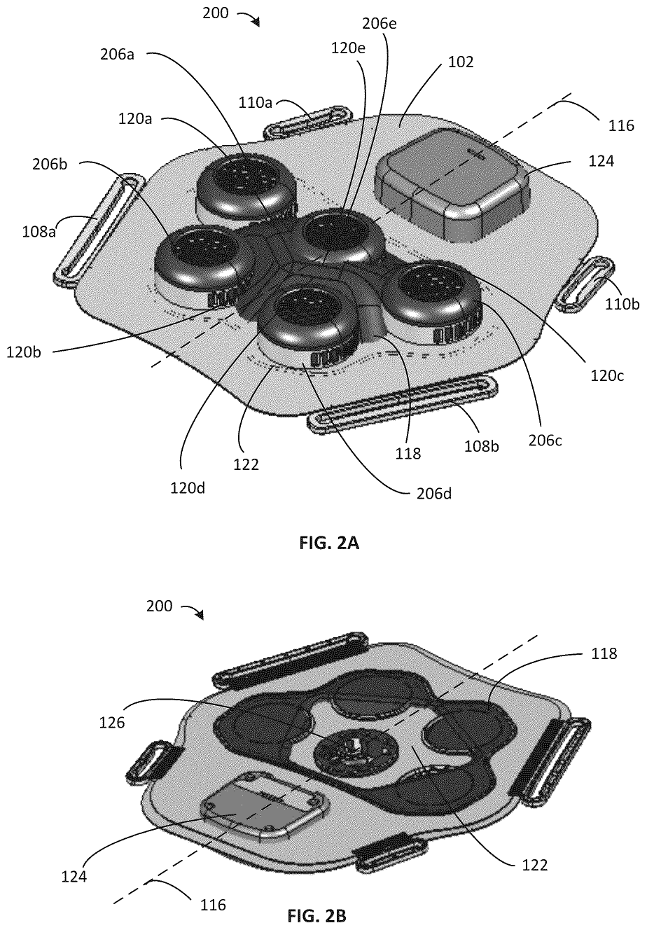

The patterns 104a, 104b can provide a structure to the apparatus 100 when placed over a shoulder. The patterns may also hide electrical wires connecting a battery in a box 124 (described below) to the cooling elements 106 and/or the controller 126, and/or the temperature modulation devices 206 (described below referring to FIG. 2A). The material for making the patterns 104, e.g., wire patterns, can be urethane.

In some embodiments, one or more cooling elements 106 may be replaced with heating-cooling elements that can selectively provide a heating or a cooling effect. In other embodiments, one or more vibration generating elements (also called vibration pods) may be provided in addition to or alongside the cooling elements 106 or the heating-cooling elements. The vibration generating elements may also provide heat. The inclusion of the vibration generating elements is optional and is not required.

The apparatus 100 is specifically designed to provide a cooling treatment to a person's shoulder. The apparatus 100 is usable for either shoulder, however. To this end, the apparatus 100 includes two large loops 108a, 108b and two small loops 110a, 110b. A relative large strap or band 112 can pass through the large loops 108a, 108b, and may be tied around the upper portion of the torso of a person. A relatively small strap or band 114 can pass through smaller loops 110a, 110b, and may be tied around the person's upper arm. The straps/bands 112, 114 may include Velcro™ fasteners, buckles, buttons, zippers, laces, hook and loop, or other types of fasteners.

When the apparatus 100 is mounted on a person's right shoulder, the pattern 104a and the cooling elements 106a, 106b may be located on the front side of the right shoulder. The pattern 104b and the cooling elements 106c, 106d may be located on the rear side of the right shoulder, and the cooling element 106e may lie over the top of the right shoulder. A portion of the pattern 104a and a portion of the pattern 104b may also lie over the top of the right shoulder.

Conversely, when the apparatus 100 is mounted on a person's left shoulder, the pattern 104b and the cooling elements 106c, 106d may be located on the front side of the left shoulder. The pattern 104a and the cooling elements 106a, 106b may be located on the rear side of the left shoulder, and the cooling element 106e may lie over the top of the right shoulder. A portion of the pattern 104a and a portion of the pattern 104b may also lie over the top of the left shoulder. Thus, regardless of the shoulder, both the patterns 104 and the cooling elements 106 are arranged symmetrically over the shoulder.

It is noted above that different embodiments may include different numbers of patterns and/or cooling elements. In general in a shoulder treatment apparatus, the number, size, and/or shape of one or more patterns and one or more cooling elements are selected such that cold treatment can be provided both to the front and back of the shoulder. In different embodiments, the respective motifs formed by the patterns and/or the cooling elements can be different from the corresponding motifs of the apparatus 100. In various embodiments, however, the apparatus 100 has an axis 116 about which the large loops 108a, 108b are symmetrical and the small loops 110a, 110b are also symmetrical. The apparatus 100 may be worn or disposed over the shoulder such that the axis substantially aligns with the collarbone of the shoulder. Thus, regardless of the particular motifs of the patterns and cooling elements in a particular embodiment, if those motifs are symmetrical about the axis 116, the patterns and/or cooling elements would be positioned generally symmetrically over the person's shoulder, and can evenly provide cold treatment to the shoulder.

In the apparatus 100, the cooling elements 106 are disposed over a base 118 of cloth or foam material having pleats 120a-120e (collectively, pleats 120). A heat conducting fabric, such as that made from polyethylene fibers, may be used to make the base 118. The cooling elements may be sewn to the base 108 or may be fastened using male-female buttons, clamps, zippers, laces, hook and loop, etc. The pleats 120 may be formed to match the geometric pattern of the cooling elements 106 such that, typically, each cooling element 106 is disposed within a respective one of the pleats 120. One advantage of such a configuration is that a pattern of the cooling elements 106, and the corresponding arrangement thereof relative to the person's shoulder, can be maintained while wearing the apparatus 100 even as the person moves, and even when the apparatus 100 is taken off and worn again at a later time.

Additionally, in some embodiments, each of the cooling elements 106 may include respective fans (not shown) to expel the heat extracted from the shoulder. The pleats 120 may help direct or divert the respective airflows from the respective fans in different respective directions. This can increase the effectiveness and/or efficiency of heat dissipation, and can improve cooling. For example, the temperature may be lowered to a level not achievable without the fans and/or the pleats, or a particular set low temperature may be achieved in a shorter time duration, than would be possible without the fans and/or pleats.

In the apparatus 100, the cooling elements 106 are disposed over and in thermal communication with a heat spreader layer 122, which is optional. A heat spreader and be a graphite or graphene heat spreader. Because all the cooling elements 106 are in thermal communication with the heat spreader layer 122, they all can extract and dissipate heat that is extracted by the heat spreader layer 122 from the shoulder. The heat spreader layer 122 can contact a greater area of the surface of the skin around the shoulder than the collective surface areas of the cooling elements 106. As such, the heat spreader layer 122 can extract more heat from the shoulder region, and/or may extract heat at a faster rate, than the cooling elements 106 may without the heat spreader layer 122. Thus, the use of the heat spreader layer 122 can increase the efficiency and/or effectiveness of providing cold treatment to the shoulder. In some embodiments, the base 118 may be made from a heat conductive material, and may function as a heat spreader, providing similar benefits as the heat spreader layer 122.

The apparatus 100 also includes a box 124 that contains a battery for powering the cooling elements 106 and a controller 126 for controlling the cooling elements 106. The battery also powers the controller 126, and can be a lithium-ion battery or a lithium-polymer battery. The box 124 can be a plastic box or a metallic box such as a Faraday cage, and is mounted to the outside surface of the apparatus 100, i.e., the surface facing away from the shoulder. In some embodiments, the box 124 includes a user interface for displaying information to and/or receiving commands from the user. A user interface may also be provided on the controller 126, in addition to or as an alternative to the user interface provided with the box 124. In some embodiments, the box 124 is not used and instead, the battery is mounted directly to the support layer 102. The controller 126 may include custom circuitry implemented using discrete components, FPGAs, ASICs, general-purpose processor(s), and/or memory to store a program or instructions to be executed by the processor.

The controller 126 may operate the cooling elements 106 such that the temperature of their respective outer contact surfaces (not shown) or that of the heat spreader layer 122 is lowered down to a set low temperature. The apparatus 100 may also include one or more temperature sensors (not shown) in thermal contact with the shoulder region. Using these sensors, the low temperatures may be regulated by the controller 126 via a feedback loop to the set low temperatures. For example, different cooling elements 106 may be set to different low temperatures, though they all can be set to the same low temperature. The low temperature values may be selected according to their respective, preset default values, according to a preset user preference, or may be set for a particular treatment session by the user. To set the low values, the user may use the user interface associated with the box 124 containing the battery or that associated with the controller 126.

In addition to controlling or regulating the cooling elements 106 according to the desired temperatures (the set low temperatures), the controller 126 may also control various timing characteristics and/or modes of the cold treatment. For example, cold treatment may be applied continuously or in a sequence, where the cooling is applied and paused intermittently, for a specified total duration or for a specified number of iterations. The iterations can be periodic or can be randomized.

The controller 126 may receive the associated timing and/or mode parameters via the user interface associated with the box 124 containing the battery or that associated with the controller 126. Example timing parameters include the duration of the cold treatments, the on and/or off durations of an iteration of a cold treatment, the total duration or the number of the iterations. Example mode parameters include whether a cold treatment is applied as a single instance, e.g., continuously, or a sequence. In some embodiments, these and/or the temperature settings may be received by the controller wirelessly, e.g., via a software application running on a smart phone, tablet, or another computer.

FIG. 2A is a perspective view of a shoulder treatment apparatus 200, and FIG. 2B is another perspective view of the apparatus 200, where certain components are removed to expose certain other components of the apparatus 200. Like the apparatus 100, the apparatus 200 also includes a support layer 102, large loops 108a, 108b, small loops 110a, 110b, the large and small straps or bands 112, 114 (not shown in FIGS. 2A and 2B), the base 118 having pleats 120, and the battery 124. Moreover, the apparatus 200 also includes the heat spreader layer 122 and the controller 126. In the apparatus 200, the patterns 104 may or may not be included. As described above, if included, they can be support structures and can hide wiring between the battery, the controller, and the temperature modulation devices (described below).

Furthermore, the cooling elements 106 of the apparatus 100 are replaced with temperature modulation devices 206a-206e (collectively, temperature modulation devices 206). In some embodiments, one or more temperature modulation devices can selectively supply heat or can provide a cooling effect. In other embodiments, one or more temperature modulation devices only provide a cooling effect, and in those embodiments, those devices may be referred to as cooling devices. In yet other embodiments, one or more temperature modulation devices only provide a heating effect, and in those embodiments, those devices may be referred to as heating devices. In some instances of the apparatus 100 (FIG. 1A), one or more cooling elements 106 may be replaced with temperature modulation devices 206 or heating devices. Although the apparatus 200 includes five temperature modulation devices 206, other embodiments may include fewer (e.g., only one, two, three, or four), or more (e.g., 6, 9, 10, etc.) temperature modulation devices. Generally, the temperature modulation devices are arranged symmetrically about an axis 116 of the apparatus 200, so that heating and/or cooling can be applied to the front and back of the shoulder, regardless of whether it is the right shoulder or the left one.

The controller 126 may control the high and/or low temperature settings and the timing and/or mode characteristics of the temperature modulation devices 206. In addition, based on a user input received from a user interface or a software application, the controller 126 may configure one or more of the temperature modulation devices 206 to provide heat treatment only, or cold treatment only, or both heat and cold treatments. In general, the controller 126 can control the voltage, polarity of the voltage, and/or current so as to control the heat generated by and/or the cooling of the temperature modulation devices 206.

Specifically, the controller 126 may heat one or more of the temperature modulation devices 206 up to a set high temperature. The controller 126 may also operate one or more of the temperature modulation devices 206 such that the temperature of their respective outer contact surfaces (not shown) or that of the heat spreader layer 122 is lowered down to a set low temperature. The apparatus 200 may also include one or more temperature sensors (not shown) in thermal contact with the shoulder region. Using these sensors, the high and/low temperatures may be regulated by the controller 126 via a feedback loop to the respective set high and/or low temperatures. The high and/or low temperature values may be selected according to their respective, preset default values, according to a preset user preference, or may be set for a particular treatment session by the user. To set the high and/or low values, the user may use the user interface associated with the box 124 containing the battery or that associated with the controller 126.

In addition to controlling or regulating one or more of the temperature modulation devices 206 according to the desired temperatures (the set high and/or low temperatures), the controller 126 may also control various timing characteristics and/or modes of the heat and/or cold treatments. For example, the heat treatment may be applied for a certain duration and the cold treatment may follow (or precede) for the same or a different duration. Each of these treatments may be applied in a sequence, where the heating and/or cooling are applied and paused intermittently, for a specified total duration or for a specified number of iterations. The iterations can be periodic or can be randomized. The heat and cold treatments may also be applied in an alternating manner.

The controller 126 may receive the associated timing and/or mode parameters via the user interface associated with the box 124 containing the battery or that associated with the controller 126. Example timing parameters include the duration of the heat and/or cold treatments, the on and/or off durations of an iteration of a heat (or cold) treatment, the total duration or the number of the iterations. Example mode parameters include whether only one kind of treatment is applied or both the heat and cold treatments are applied, whether a heat or cold treatment is applied as a single instance or a sequence, and whether the two treatments are applied in an alternating manner. In some embodiments, these and/or the temperature settings may be received by the controller wirelessly, e.g., via a software application running on a smart phone, tablet, or another computer.

Referring collectively to FIGS. 1A, 1B, 2A, and 2B, in some embodiments, the heating and/or cooling apparatuses 100, 200 may include a bladder that can be used to apply compression against at least a region of the shoulder while providing heating and/or cooling (and optionally vibrational) therapy. An example of a temperature therapy device having a blader is disclosed in U.S. Patent Application Publication No. 2022/0110821, titled “System for Mounting Inelastic Components to a Flexible Material to Apply Compressive and Thermal Therapy,” which is incorporated herein by reference in its entirety. The bladder can be an inflatable air bladder.

Referring again to FIGS. 1A and 2A, the bladder can be disposed over the cooling devices 106 or the temperature modulation devices 206, and the box 124, according to some embodiments. The bladder can push these devices and/or the support layer 102 towards the shoulder region, and adjust the shape of the support layer 102 and/or the positions of the devices 106, 206 according to the contours of the shoulder region. The apparatuses 100, 200 can then wrap over the shoulder tightly. This can provide a firmer physical contact between the shoulder region and the outer, exposed surfaces (not shown) of the apparatuses 100, 200 and/or the cooling devices 106 or the temperature modulation devices 206. The firmer contact can increase the effectiveness of the delivery of heat to the shoulder region and/or the extraction of heat from the shoulder region, and thus the effectiveness of the cooling treatment. The bladder can also provide a therapeutic compressive force to the shoulder region.

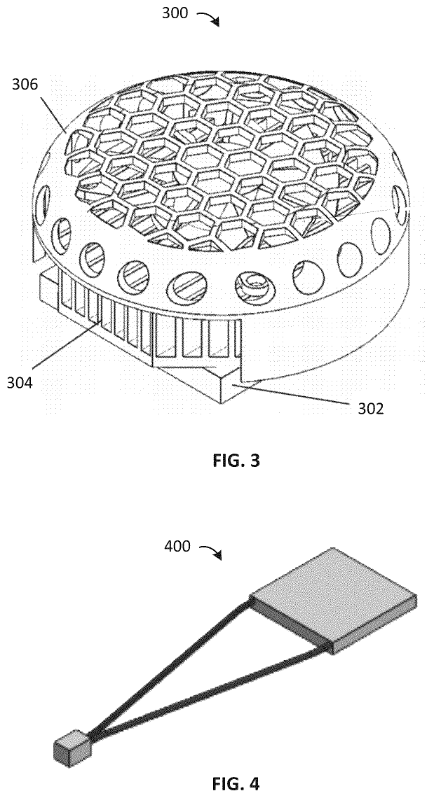

FIG. 3 discloses a temperature modulation device 300 that may be used in the apparatuses 100, 200 (FIGS. 1 and 2). The temperature modulation device includes a temperature controllable layer 302 (e.g., temperature-controllable substrate), a heat exchanger 304, and a shield 306. The heat exchanger 304 may includes a passive heat transfer element and/or an active heat transfer element.

The temperature controllable layer 302 generally functions to provide a thermomechanical interface through which heat can be exchanged with the skin region of a user. To this end, the temperature controllable layer 302 provides a “contact surface” (e.g., proximal surface) that is maintained at a set temperature (e.g., set by the control module, set by the user at a client application in communication with the control module, etc.). The contact surface can have any suitable shape, but is preferably a broad surface. As used herein, the “contact surface” is in thermal contact with the skin of the treatment region, but may or may not be in physical contact with the skin. Thus, the heat exchange through the contact surface can be direct or through one or more layers, such as the base 118 and/or the heat spreader layer 122 (FIG. 1A). The temperature controllable layer 302 also provides an opposing a non-contact surface (e.g., distal surface). When the temperature modulation device 300 is operated as a cooling device, the non-contact surface generally functions as the surface at which the heat extracted from the skin region is dissipated. When the temperature modulation device 300 is operated as a heating device, heat may be absorbed through the non-contact surface, so that it may be delivered to the skin through the contact surface. The temperature controllable layer 302 may optionally include an interface layer, such as a thermal pad, a gel layer, a graphite layer, etc.

In some embodiments, the temperature modulation device 300 includes a thermoelectric cooling and/or heating device (e.g., a Peltier cooler and/or heater, any other suitable type of thermoelectric cooler/heater or panel, etc.). In such a device, typically, an applied voltage generates a temperature differential between the contact and non-contact surfaces that is based on the applied voltage. A temperature modulation device 300 may include, for example, a Peltier thermoelectric module having a square cross section of 40 mm×40 mm (or any other suitable footprint having different sizes and/or shapes), and having a thickness of 4.2 mm (or any other suitable thickness). The Peltier thermoelectric module may be adapted to receive a range of currents (e.g., between 0.5-2 A) at a specified voltage (e.g., approximately 15 V) that can be reversed in polarity in order to generate either a high temperature (e.g., 100-120° F.) or a low temperature (e.g., 40-60° F.) at the contact surface. Examples of such thermoelectric modules include thermoelectric coolers (TECs): TEC1-12702, TEC1-12703, TEC1-12701, TEC1-12706, or any other suitable thermoelectric module. Appendix A provides the details of a TEC.

FIG. 4 schematically depicts a thermoelectric cooler (TEC) 400. The TEC 400 can be selected based on its thermal conductivity rating. For example, a TEC 400 may have a high thermal conductivity rating, e.g., greater than or equal to the thermal conductivity of a ceramic material. The TEC 400 is square shaped and has a size of approximately 40 mm×40 mm. Rectangular TECs or those having other different shapes (e.g., circular, ovular, hexagonal, other polygonal, etc.) may also be used in different embodiments. A thermal grease can be disposed between a heat spreader layer (e.g., the heat spreader layer 122 or the base 118 of FIG. 1A) and the TEC 400. A thermal grease having a high thermal conductivity, e.g., in the range of approximately 1-15 w/mk can be used.

Referring back to FIG. 3, in some embodiments, the temperature controllable layer 302 defines an internal void (e.g., a hollow interior of the layer, a set of tubes, etc.) through which a circulating fluid is pumped by a pumping mechanism. The circulating fluid may be heated and/or cooled to a controlled temperature, e.g., a set high temperature, a set low temperature, etc., so as to provide a heating or a cooling effect.

When the temperature modulation device 300 is operated as a cooling device, the heat exchanger 304 may transport heat from temperature controllable layer 302 to the ambient environment surrounding the overall system containing the device 300. Thus, the heat exchanger 304 may dissipate the heat extracted from the user's body into the temperature controllable layer 302. A passive heat transfer element that may be included in the heat exchanger 304 can be a heat sink. An active heat transfer element that may be included in the heat exchanger 304 can be a fan (e.g., a rotary fan). Other types of heat transfer elements (e.g., a circulating-fluid heat exchanger, an evaporative heat exchanger, a condenser, etc.), are also contemplated. Appendix B describes additional details of different embodiments of a temperature modulation device.

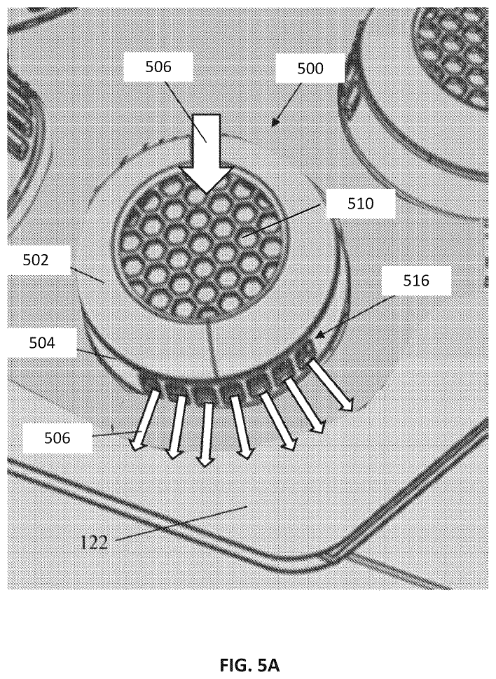

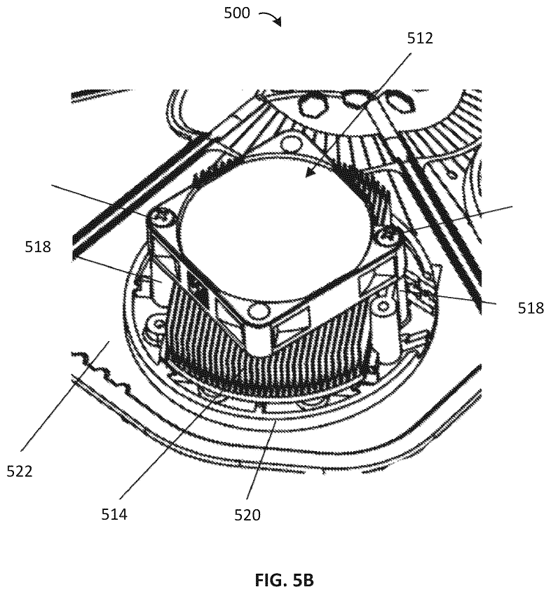

FIG. 5A is a perspective view of another temperature modulation device 500 that may be used in the apparatuses 100, 200 (FIGS. 1 and 2). The temperature modulation device 500 include a cap 502 and cover 504. FIG. 5B is a top view of the temperature modulation device 500 without the cover and cap. Referring to FIG. 5A, The direction of the air flow through the device 500 is shown by the arrows 506. The air flow can help regulate the temperature of the mounting plate 508 (FIG. 5B). The cap 502 has an air inlet 510 through which a fan 512 pulls air into a heatsink 514 (FIG. 5B). Heated air from the heatsink 514 may exit or may be forced out through the vents 516. In some embodiments, the air flow is reversible, whether the air is pulled in from the vents 516 and expelled from the inlet 510.

Referring to FIG. 5B, the temperature modulation device include a spacer 518 having a mounting plate 520. A heat spreader layer 522 may be attached, e.g., using an adhesive, to the bottom of the mounting plate 520. The heat spreader layer 522 can be the same as the heat spreader layer 122 or the base 118 of the apparatus 100 (FIG. 1A). In some embodiments, the heat spreader 522 is thermally coupled with the skin of the shoulder region via an optional thermally conductive protective layer.

In some embodiments that are otherwise similar to the apparatus 100 (FIG. 1A) or the apparatus 200 (FIG. 2A), one or more cooling elements 106 or temperature modulation devices 206 are replaced with a vibration generating device. Such devices can apply rapid, percussive, tapping forces to the shoulder region, e.g., to massage the shoulder, and can also apply heat. The use of vibration generating devices is optional, however, and not required. The controller 126 may be configured to control various parameters of the vibration device, such as the frequency and/or intensity (force) of vibrations, and whether the vibrations are applied continuously or in a sequence. In the latter case, the controller 126 may also control the on and/or off durations of the iterations in a sequence. Moreover, the controller 126 may be configured to apply the heat before, during, or after the percussive, tapping forces are applied.

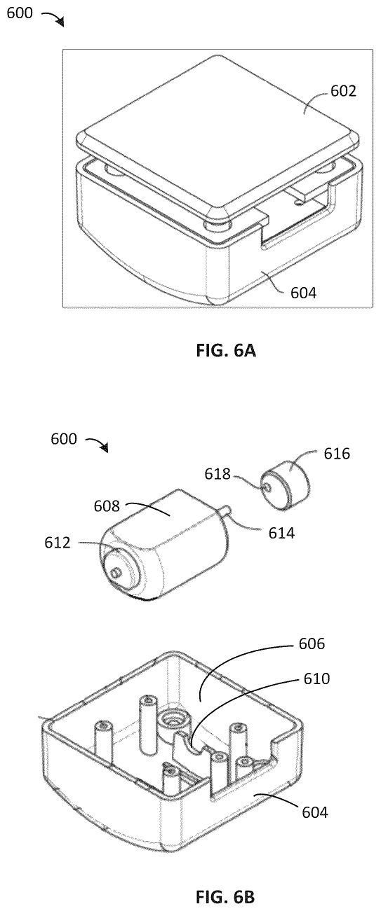

FIG. 6A depicts a vibration generation device 600. FIG. 6B is a partial exploded view of the device 600. The device 600 has a top surface 602 and a bottom cover 604, that provides a cavity 606. A motor 608 is disposed in the cavity 606 over support rims 610 (only one is shown). The motor 608 has a rear bearing 612 and a front bearing (not shown) mounted to a shaft 614. A mass 616 has an off-center bore 618, and is mounted to the motor 608 such that the shaft 614 engages with the bore 618. As such, when the motor 608 is operated, the mass 616 rotates about the shaft 614 eccentrically. This causes the shaft 614 to vibrate, and these vibrations are transferred to the cover 604 through the support rims 610. The device 600 is disposed in the apparatus 100 (FIG. 1A) such that the outer surface of the cover 604 is placed in physical contact with the skin of the shoulder region to be treated. The physical contact can be direct or via one or more layers. As such, the vibrating cover 604 may be deliver a vibrating force to the shoulder region.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Particular embodiments of the subject matter have been described. Other embodiments are within the scope of the following claims. Accordingly, other implementations are within the scope of the following claims. The phraseology and terminology used herein is for the purpose of description and should not be regarded as limiting.

The term “approximately”, the phrase “approximately equal to”, and other similar phrases, as used in the specification and the claims (e.g., “X has a value of approximately Y” or “X is approximately equal to Y”), should be understood to mean that one value (X) is within a predetermined range of another value (Y). The predetermined range may be plus or minus 20%, 10%, 5%, 3%, 1%, 0.1%, or less than 0.1%, unless otherwise indicated.

The indefinite articles “a” and “an,” as used in the specification and in the claims, unless clearly indicated to the contrary, should be understood to mean “at least one.” The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The use of “including,” “comprising,” “having,” “containing,” “involving,” and variations thereof, is meant to encompass the items listed thereafter and additional items.

Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

As various changes could be made in the above constructions without departing from the scope of the invention, it is intended that all the matter contained in the above description or shown in the accompanying drawings shall be interpreted as illustrative and not in a limiting sense.

What is claimed is:

1. An apparatus for providing thermal therapy to a shoulder, the apparatus comprising:
a support layer having an axis alignable with a collarbone of a shoulder;
a plurality of temperature modulation devices mounted to the support layer in a symmetrical pattern about the axis; and
a controller for controlling a temperature of at least one of the temperature modulation devices.

2. The apparatus of claim 1, wherein the plurality of temperature modulation devices comprises a plurality of cooling elements.

3. The apparatus of claim 2, wherein at least one of the plurality of cooling elements comprises a thermoelectric cooling (TEC) device.

4. The apparatus of claim 1, wherein:
the plurality of temperature modulation devices comprises: (i) a first plurality of heating elements, and (ii) a second, different plurality of cooling elements.

5. The apparatus of claim 4, wherein at least one of the plurality of cooling elements comprises a thermoelectric cooling (TEC) device.

6. The apparatus of claim 1, wherein at least one of the plurality of temperature modulation devices comprises a heating-cooling device that can selectively provide heat or a cooling effect.

7. The apparatus of claim 1, further comprising:
a base having a plurality of pleats, wherein at least a subset of the plurality of temperature modulation devices are disposed in respective pleats of the plurality of pleats.

8. The apparatus of claim 1, further comprising:
a battery for supplying electrical power to the controller and to at least one of the plurality of temperature modulation devices.

9. The apparatus of claim 1, further comprising:
a first set of loops and an associated first strap wrappable around a torso; and
a second set of loops and an associated second strap wrappable around an upper arm, so as to substantially align the axis of the support layer with the collarbone of a shoulder.

10. The apparatus of claim 1, further comprising:
a plurality of vibration generation devices mounted to the support layer in a symmetrical pattern about the axis.
